(19) United States
(12) Patent Application Publication
(10) Pub. No.: US 2025/0032978 A1
(43) Pub. Date: Jan. 30, 2025

(54) Device and Method for the Sequestration of Atmospheric Carbon Dioxide

(71) Applicant: UB - ONE STIFTUNGS GMBH, Heidelberg (DE)

(72) Inventor: Jan-Heiner Küpper, Kleinkoschen (DE)

(73) Assignee: UB - ONE STIFTUNGS GMBH, Heidelberg (DE)

(21) Appl. No.: 18/914,793

(22) Filed: Oct. 14, 2024

Related U.S. Application Data
(63) Continuation of application No. 18/295,041, filed on Apr. 3, 2023, now abandoned.
Application No. 18/295,041 is a continuation of application No. 16/761,313, filed on May 4, 2020, now abandoned.

(30) Foreign Application Priority Data
Nov. 4, 2017 (EP) 17200037.4

(51) Int. Cl. B01D 53/62; B01D 19/00; B01D 53/04; B01D 53/053; B01D 53/85; C12M 1/00; C12N 1/12

(52) U.S. Cl. CPC B01D 53/62; B01D 19/0057; B01D 53/0462; B01D 53/053; B01D 53/85; C12M 21/02; C12M 43/00; C12N 1/12; B01D 2251/95; B01D 2257/504; B01D 2259/802

(57) ABSTRACT

The invention relates to a device and to a method for sequestering atmospheric carbon dioxide using at least one air capture module in conjunction with a biorcactor equipped with an autotrophic microorganisms.

### CROSS-REFERENCE TO RELATED APPLICATIONS

This application is a continuation of U.S. patent application Ser. No. 18/295,041 filed on Apr. 3, 2023, which is a continuation of U.S. patent application Ser. No. 16/761,313 filed on May 4, 2020, which is a U.S. National Phase of International Patent Application No. PCT/EP2018/080134, filed on Nov. 5, 2018, which claims priority to European Application Serial No. 17200037.4 filed Nov. 4, 2017. The entire contents of these applications are incorporated herein by reference in their entirety.

### BACKGROUND

The invention relates to a device and to a method for sequestering atmospheric carbon dioxide using an air capture module in functional conjunction with a bioreactor equipped with autotrophic microorganisms.

The need to quantitatively sequester carbon dioxide (CO₂) from the atmosphere is viewed as a global problem. In addition to significantly reducing the use of fossil fuels, a direct sequestration of CO₂ from the atmosphere is considered to be necessary to be able to achieve the worldwide climate targets. These consist in a maximum permissible temperature increase of less than 2° C. compared to when record-keeping began. Other measures, such as geoengineering, for example by iron fertilization of the ocean or the introduction of sulfur compounds into the atmosphere so as to enhance the reflection of solar radiation, are rated as very risky, with ecological consequences.

According to the findings of climate researchers, an average global temperature rise of more than 2° C. results in irreversible disruptions of the climate systems. Another global problem is the decarbonization of industry, which often accompanies the phase-out of the use of fossil fuels and energy sources. This means that carbon sources other than fossil sources have to be found for chemical processes.

Technology describes the direct sequestration of carbon dioxide from the atmosphere using bioenergy with carbon capture and storage (BECCS). This involves using cultivated crops for energy purposes (biomass and gas-fired power plants) and storing the arising CO₂ in geological strata. BECCS, however, has the following drawbacks: 1.) CO₂ injection into geological strata, which is associated with risks and only possible in few regions of the earth. 2.) Competition with agriculture since the high land requirement for BECCS results in a shortage of cultivation space for food production.

The use of photobioreactors, which contain autotrophically growing microorganisms and produce biomass, is considered to be a promising option of carbon dioxide sequestration. For example, it is easy to use microalgae. This biomass has a variety of uses, such as 1.) biogas generation for energy production, 2.) recovery of carbon compounds for the chemical industry, 3.) biofuels, and 4.) food additives, which can be contained especially in algae, 5.) other valuable substances such as pharmaceutically acting substances and cosmetics, 6.) organic fertilizer made of biomass (biofertilizer).

In the prior art, WO 1998/045409 A1 and EP 2 568 038 A1 describe laminar photobioreactors for the production of microalgae, wherein the following problems are discussed:
a.) A suitable microorganism has to be used, which is easy and inexpensive to cultivate and has a high biomass production.
b.) A continuous CO₂ supply must be ensured since the atmospheric CO₂ concentration in the amount of 400 ppm (0.04%) does not allow optimal growth of microalgae, for example. It was found that, at optimal CO₂ concentrations, microalgae create biomass approximately 10 to 50 times more efficiently than crops. The technical teaching describes that microalgae such as Chlorella, Scenedesmus, Spirulina, Nannochloropsis, Nostoc and Chlorococcus are able to grow very well in the range of 1 to 20% CO₂ (that is approximately 25 to 500 times higher than in the atmosphere), and have an accordingly high biomass productivity (see also Appl. Biochem. Biotechnology, 2016 179:1248-1261 and the literature cited therein). Previously, the problem was solved by using chemically pure CO₂ (technical CO₂). Of course, this does not solve the problem of carbon sequestration since this CO₂ is obtained in a highly energy-consuming process as a by-product in the chemical industry. A variety of working groups have already attempted to use alternatives in the form of waste gas flows from power plants. Even though this would allow the CO₂ arising during the combustion of fossil energy sources to be sequestered, it would not ensure a direct removal of CO₂ from the atmosphere. Moreover, it is known that waste gas flows from power plants contain impurities such as sulfur, nitrogen oxides, carbon monoxide and heavy metals, which can drastically inhibit the growth of microorganisms. Removing harmful impurities from these waste gas flows is a very cost-intensive process. In contrast, a direct introduction of atmospheric air into photobioreactors would have the drawbacks that, first, too little CO₂ is present for optimal growth and, secondly, that algae predators such as protozoa and zooplankton may be present on small dust particles in the air. These organisms subsist on algae and can thus heavily disrupt the bioreactor operation.
c.) For microalgae to grow optimally, it is necessary that the oxygen that develops during the light reaction is removed since it can have a toxic effect, and moreover also triggers the process of photorespiration, wherein CO₂ is formed again.
d.) Another problem is that an efficient bioreactor should allow a continuous operation, that is, the supply of nutrient solution and removal of biomass take place constantly, without having to stop the reactor. Moreover, a bioreactor should be configured so flexibly that different types of microalgae, and even prokaryotic chemolithotrophic CO₂ fixers, can be cultivated.
e.) The photobioreactor has to maintain optimal growth conditions of the microorganism, such as temperature, pH value, nutrients and the like.

The prior art, however, does not describe a suitable device and method for sequestering atmospheric carbon dioxide using a bioreactor, and in particular a photobioreactor.

It is therefore an object of the invention to provide a suitable device or a method for sequestering atmospheric carbon dioxide by producing biomass.

### SUMMARY OF THE INVENTION

In an aspect, the invention is directed to a device for sequestering atmospheric carbon dioxide, the device comprising at least one module comprising a capture unit configured for binding the atmospheric carbon dioxide by way of an adsorber material and, after treatment by way of heat or a vacuum, the atmospheric carbon dioxide being kept available, and the at least one module being connected to at least one bioreactor, wherein the atmospheric carbon dioxide is continuously supplied to autotrophic microorganisms in the at least one bioreactor.

In another aspect, the invention is directed to a device for sequestering atmospheric carbon dioxide, the device comprising a module comprising a capture unit, wherein the atmospheric carbon dioxide is bound by way of an adsorber material and, after treatment by way of heat or a vacuum, the atmospheric carbon dioxide is kept available in a pressurized container, and at least one bioreactor containing autotrophic microorganisms.

In another aspect, the invention is directed to a method for sequestering atmospheric carbon dioxide, at least one module comprising a capture unit configured for binding the atmospheric carbon dioxide by way of an adsorber material and, after treatment by way of heat or a vacuum, the atmospheric carbon dioxide being kept available, and the at least one module being connected to at least one bioreactor, wherein the atmospheric carbon dioxide is continuously supplied to autotrophic microorganisms in the at least one bioreactor.

### BRIEF DESCRIPTION OF THE FIGURES

FIG. 1A shows an exemplary embodiment of a device for sequestering atmospheric carbon dioxide according to the invention.

FIG. 1B shows another exemplary embodiment of a device for sequestering atmospheric carbon dioxide according to the invention.

### DETAILED DESCRIPTION OF THE INVENTION

It is an object of the invention to provide a suitable device or a method for sequestering atmospheric carbon dioxide by producing biomass.

To achieve this object, the invention thus relates to a device for sequestering atmospheric carbon dioxide, wherein at least one module comprising a capture unit binds atmospheric carbon dioxide by way of an adsorber material and, after treatment by way of heat or a vacuum, the atmospheric carbon dioxide is kept available, and the module is connected to at least one bioreactor, wherein atmospheric carbon dioxide is continuously supplied to autotrophic microorganisms in at least one bioreactor.

In another preferred embodiment, the invention relates to a device for sequestering atmospheric carbon dioxide, wherein at least one module comprising a capture unit binds atmospheric carbon dioxide by way of an adsorber material and, after treatment by way of heat or a vacuum, the atmospheric carbon dioxide is kept available in a container, in particular a pressurized container, wherein atmospheric carbon dioxide is continuously supplied to autotrophic microorganisms in at least one bioreactor.

In another embodiment, the invention relates to a device for sequestering atmospheric carbon dioxide, comprising a module comprising a capture unit, wherein atmospheric carbon dioxide is bound by way of an adsorber material and, after treatment by way of heat or a vacuum, the atmospheric carbon dioxide is kept available in a pressurized container, and at least one bioreactor containing autotrophic microorganisms.

A pressure reducer can be assigned to such a pressurized container, so that a continuous CO₂ stream can be provided, if necessary using measuring and control technology.

In another preferred embodiment, atmospheric carbon dioxide can be supplied to autotrophic microorganisms in at least one bioreactor together with air. Ratios of 5:95 vol. % CO₂/air, and in particular from 1:99 vol. % CO₂/air to 10:90 vol. % CO₂/air, are preferred.

To achieve this object, the invention thus likewise relates to a method for sequestering atmospheric carbon dioxide, wherein at least one module comprising a capture unit binds atmospheric carbon dioxide by way of an adsorber material and, after treatment by way of heat or a vacuum, the atmospheric carbon dioxide is kept available, and the module is connected to at least one bioreactor, wherein atmospheric carbon dioxide is continuously supplied to autotrophic microorganisms in at least one bioreactor.

The prior art describes the sequestration of CO₂ from industrial waste gases by way of a bioreactor, which, however, is entirely different, since such waste gases are of a different quality and, air contains other harmful substances and has an insufficient CO₂ concentration.

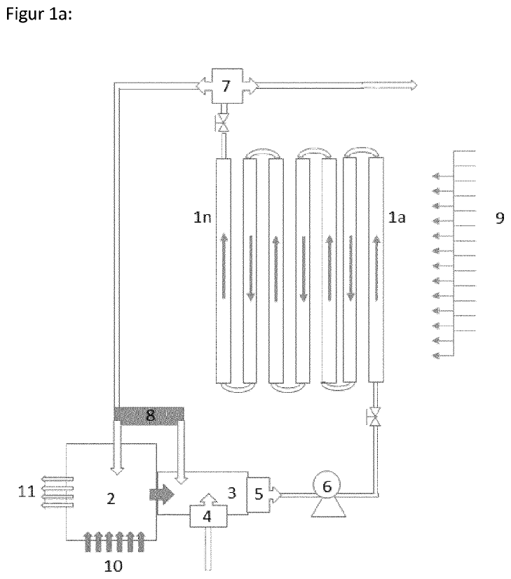

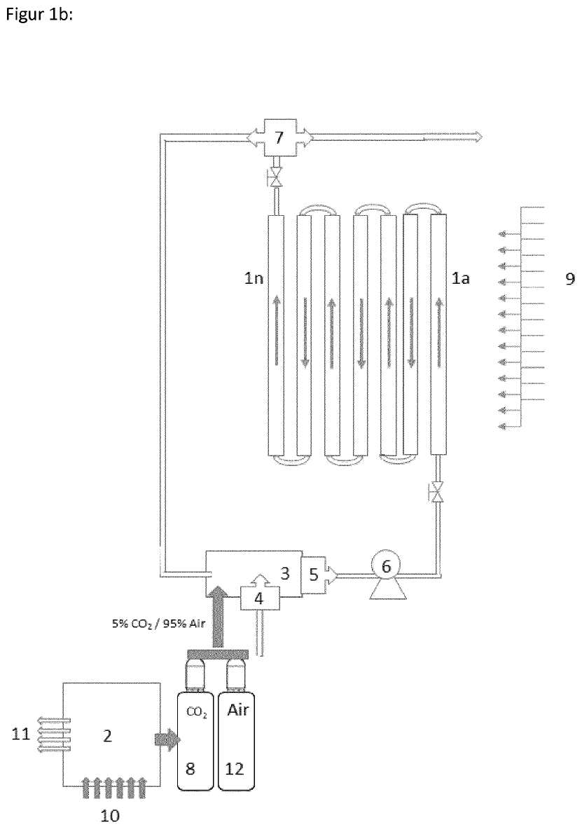

In a preferred embodiment, the device according to the invention includes such features according to FIG. 1a or FIG. 1b, whereby the above-described problems can be solved completely for the first time.

Preferably, bioreactor modules that run parallel and are connected to one another are used (1a-1n, FIGS. 1a, 1b). These are fed a nutrient solution including the autotrophic microorganism to be cultivated, preferably microalgae of the genus chlorella, Scenedesmus, Spirulina, Nannochloropsis, Nostoc or Chlorococcus (3, FIGS. 1a, 1b). Chemically pure CO₂ is introduced into the nutrient solution, preferably together with air, wherein the CO₂ preferably stems from a connected air capture module (carbon dioxide recovery installation) (2, FIGS. 1a, 1b). In particular, the aforementioned algae exhibit favorable growth rates in the device according to the invention, including the method according to the invention that is carried out.

The company Climeworks in Switzerland (climeworks.com) produces functional air capture modules, for example, which can be connected to the bioreactor in accordance with the invention. Atmospheric CO₂ is bound by way of these air capture modules (10, FIGS. 1a, 1b) and can subsequently be released again by way of heating at approximately 100° C. In contrast, atmospheric oxygen or nitrogen is not bound, but is returned into the atmosphere (11, FIGS. 1a, 1b). By combining the air capture module with a bioreactor, it is achieved for the first time that atmospheric CO₂ is pre-concentrated in a form that is optimal for microorganisms, without additional interfering components, such as harmful substances or algae predators, being present. The latter are efficiently destroyed by the heating process for CO₂ release.

A measuring and control unit (5, FIGS. 1a, 1b) measures critical parameters such as the CO₂ concentration, pH value, algae biomass per unit of volume. Thereafter, the solution is transferred into the bioreactor via a system pump (6, FIGS. 1a, 1b). In the case of photobiorcactors, illumination takes place (9, FIGS. 1a, 1b). As a result of the translucency of the material, the preferred algae according to the invention, serving as the microorganism, are able to carry out photosynthesis. The optimal CO₂ concentration, which can be flexibly set by way of the air capture module, causes considerable reproduction in the reactor modules. Algae biomass can, on the one hand, be given off continuously via a central measuring and control unit (7, FIGS. 1a, 1b) and processed by way of common methods.

On the other hand, this is preferably a continuous bioreactor, which can operate in a circuit. The algae are conducted across a vapor-liquid separator (also: gas-liquid separator) (8, FIG. 1a). The principle of gas separation from a photobioreactor operated with microalgae is known. For example, the algae can be conducted through a chamber containing a semipermeable membrane, by which the gases (O₂/CO₂) present in the liquid are removed by way of diffusion. Another technical solution is the use of a mechanical, vortex-driven gas separator (Fasoulas et al., University of Stuttgart, status report on the 2nd preliminary result within the scope of the project 50 JR 1104 “Regenerative Lebenserhaltungssysteme für die Raumfahrt mit synergetisch integrierten Photobioreaktoren und Brennstoffzellen (Regenerative life-sustaining systems for the aerospace industry with synergetically integrated photobioreactors and fuel cells)” funded by the DLR space agency in the time period, 2014). The gas (oxygen and unconsumed CO₂) is returned into the air capture module via the separator (2, FIGS. 1a, 1b). In the process the O₂ escapes, wherein the CO₂ is bound again and conducted into the circuit. This advantageously solves the problem of the continuous removal of O₂. The algae are conducted from the vapor-liquid separator into the central cultivation tank again (3, FIGS. 1a, 1b). Here, the CO₂ concentration can now be set to the optimal value again, and nutrient solution can be supplied from outside (4, FIGS. 1a, 1b).

The invention thus relates to such a device according to the invention which additionally comprises a gas-liquid separator, so that a continuous circulatory process can advantageously be achieved, and arising oxygen can be removed.

In another preferred embodiment, 5 to 50% of the culture medium or nutrient solution is replaced within a day. The device comprises a measuring unit (7, FIG. 1a), for example, which opens a faucet at a defined biomass concentration (for example, 1 g/liter, measured by way of the optical density (OD650ₙₘ) of the medium) so as to conduct a defined proportion of the culture medium into a collection vessel. At the same time, the missing and fresh culture volume (4, FIG. 1a) is supplied again.

The installation can likewise be operated with chemo (litho) autotrophic bacteria, such as Archaea bacteria, which likewise receive CO₂ via the air capture module. A light reaction is not required, but an energy source in the form of H₂ (molecular hydrogen) is.

NaHCO₃ (4.05×10⁻² M), Na₂CO₃ (9.50×10⁻³ M), K₂HPO₄ (7.17×10⁻⁴ M), NaNO₃ (7.35×10⁻³ M), K₂SO₄ (1.43×10⁻³ M), NaCl (4.27×10⁻³ M), MgSO₄×7H₂O (4.15×10⁻⁴ M), CaCl₂)×2H₂O (9.01×10⁻⁵ M), FeSO₄×7H₂O (1.64×10⁻⁵ M), EDTA=Titriplex III (0.04 g/L)+2.5 ml/L micro nutrient medium (2.2 mg/L ZnSO₄×7H₂O, 25 mg/L MnSO₄×4H₂O, 28 mg/L H₃BO₃, 2 mg/L Co[NO₃]2×6H₂O, 0.21 mg/L Na₂ MoO₄×2H₂O, 0.79 mg/L CuSO₄×5H₂O)+1 ml/L Vitamin B12 (1.5 g/L). The pH value is 9.3.

Initially, a sterile starter culture (1 L) is inoculated with Spirulina platensis (Culture Collection of Algae Göttingen, SAG) in the above-described nutrient solution in a shake flask (shake frequency of 100 to 120 rpm) and cultivated in the batch for 3 to 4 days. The photon flux density (PFD) is set to 100 to 150 μmol/m²s. The gasification is carried out by way of a cotton stopper and diffusion.

The flat plate photobioreactor is inoculated with this starter culture, and the entire system (see FIG. 1) is put into operation. It is gasified with a mixture of 5% CO₂/air. The medium is preferably moved by way of a system pump, or the medium can also be circulated by way of a membrane-assisted so-called air-lift technique. The temperature of the nutrient medium in the reactor is preferably 30° C.

The installation is designed so as to be operable in a batch process, that is, the biomass is only harvested once at the end of the experiment. In this case, the bioreactor is operated for 5 to 8 days. The highest productivity, however, is preferably achieved during continuous or semi-continuous operation. A defined proportion of the reactor volume is replaced with fresh culture medium or nutrient medium in the process (see devices 4 and 7 in FIG. 1a). The highest productivity is achieved when 30% of the nutrient medium is replaced every day. In the batch process, the productivity is, on average, 500 to 800 mg algae biomass/liter/day. By continuously replacing the nutrient medium (30% per day), a productivity of 1.5 g algae biomass/liter/day is achieved.

Algae biomass using open pond bioreactor (Appl Microbiol Biotechnol (2007) 74:1163-1174)): Instead of the flat plate photobioreactor, an open system is used, which has a volume of 500 L. The nutrient medium (see above) is continuously circulated using a flow rate of 0.2 to 0.5 m s⁻¹ by way of electrically operated bucket wheel-like paddles. The open pond system is operated in a batch process or in a semi-continuous process. After inoculation with 10 liters of spirulina starter culture (see above), the cultivation is carried out in a batch process up to 7 days. In the semi-continuous process, a certain proportion (for example 10%) of the medium in which the microalgae have multiplied is harvested every day, and replaced with new medium. The open pond system is illuminated in a closed space from above using LEDs of the BX180 series (Valoya, Finland). The open pond system is gasified with a 2.5% CO₂/air mixture. The CO₂ is provided by way of an air capture module. The room temperature is 24° C. After seven days, the biomass is harvested or the bioreactor is run on a semi-continuous basis. The concentration of the biomass is approximately 5 g/L.

### Example 5

### Example of Carbon Sequestration by Way of Humus Formation

One of the following microalgae capable of nitrogen fixation is inoculated in the closed photobioreactor or in the open pond system with CO₂ supply (mixture of 2.5% CO₂ and air): Nostoc, Anabaena, Aulosira, Tolypothrix, Nodularia, Cylindrospermum, Scytonema, Aphanothece, Calothrix, Anabaenopsis, Mastigocladus, Fischerella, Stigonema, Haplosiphon, Chlorogloeopsis, Camptylonema, Gloeotrichia, Nostochopsis, Rivularia, Schytonematopsis, Westiella, Westiellopsis, Wollea, Plectonema, Chlorogloea.

Nostoc muscorum is well-suited for the open pond system and grows in liquid medium in a manner similar to spirulina. Nostoc muscorum is cultivated for 14 days and then harvested as a batch. As an alternative, a semi-continuous cultivation is carried out, wherein every day approximately 10% of the resultant biomass is harvested, and the withdrawn medium is replaced with fresh culture medium. During the cultivation phase, atmospheric nitrogen is fixed by the algae. The algae biomass is dried. The batch process results in a yield of 700 mg biomass/L. The dry biomass is pressed to form granules, which are distributed in the soil as biofertilizer. This algae biomass is largely composed of carbon (>50%), which stems from the CO₂ fixation in the case of autotrophic growth. The inoculation of a suitable soil substrate with Nostoc also results in an improvement in the supply of nitrogen. The biomass has a ratio of carbon to nitrogen of 10 to 15:1.

The biofertilizer made of algae biomass improves the growth of plants, such as trees, whereby further CO₂ sequestration is enabled.

1. A device for sequestering atmospheric carbon dioxide, the device comprising at least one module comprising a capture unit configured for binding the atmospheric carbon dioxide by way of an adsorber material and, after treatment by way of heat or a vacuum, the atmospheric carbon dioxide being kept available, and the at least one module being connected to at least one bioreactor, wherein the atmospheric carbon dioxide is continuously supplied to autotrophic microorganisms in the at least one bioreactor.

2. The device for sequestering atmospheric carbon dioxide according to claim 1, wherein the atmospheric carbon dioxide is kept available in a container.

3. A device for sequestering atmospheric carbon dioxide, the device comprising
a module comprising a capture unit, wherein the atmospheric carbon dioxide is bound by way of an adsorber material and, after treatment by way of heat or a vacuum, the atmospheric carbon dioxide is kept available in a pressurized container, and
at least one bioreactor containing autotrophic microorganisms.

4. The device for sequestering atmospheric carbon dioxide according to claim 1, further comprising at least one gas-liquid separator.

5. The device for sequestering atmospheric carbon dioxide according to claim 1, wherein the at least one bioreactor is a photobioreactor or an open pond bioreactor.

6. The device for sequestering atmospheric carbon dioxide according to claim 1, wherein the at least one module is an air capture module.

7. The device for sequestering atmospheric carbon dioxide according to claim 1 wherein the autotrophic microorganisms are photoautotrophic microorganisms, or chemoautotrophic microorganisms.

8. The device for sequestering atmospheric carbon dioxide according to claim 1, wherein the atmospheric carbon dioxide is supplied to the autotrophic microorganisms in the at least one bioreactor together with air.

9. The device for sequestering atmospheric carbon dioxide according to claim 1, wherein 5 to 50% of a culture medium in the at least one bioreactor is replaced.

10. A method for sequestering atmospheric carbon dioxide, at least one module comprising a capture unit configured for binding the atmospheric carbon dioxide by way of an adsorber material and, after treatment by way of heat or a vacuum, the atmospheric carbon dioxide being kept available, and the at least one module being connected to at least one bioreactor, wherein the atmospheric carbon dioxide is continuously supplied to autotrophic microorganisms in the at least one bioreactor.

11. The method for sequestering atmospheric carbon dioxide according to claim 10, wherein the at least one bioreactor is operated continuously.

12. A method for sequestering atmospheric carbon dioxide from ambient air, the method comprising utilizing the device according to claim 1.

13. The device according to claim 2, wherein the container is a pressurized container.

14. The device for sequestering atmospheric carbon dioxide according to claim 7, wherein the autotrophic microorganisms are of the genus Chlorella.

15. The device for sequestering atmospheric carbon dioxide according to claim 8, wherein a ratio of carbon dioxide to air is from 1:99 vol. % CO₂/air to 10:90 vol. % CO₂/air.

16. The device for sequestering atmospheric carbon dioxide according to claim 7, wherein the autotrophic microorganisms are selected from the group consisting of Archaea bacteria, algae, micro algae, Chlorella, Scenedesmus, Spirulina, Nannochloropsis, Nostoc, and Chlorococcus.
